(19) United States
(12) Patent Application Publication
(10) Pub. No.: US 2007/0105942 A1
(43) Pub. Date: May 10, 2007

(54) Supplemental dietary composition for turning on anabolic switches in muscle, stimulating and/or optimizing protein synthesis, and/or potently signaling muscle building and/or growth via molecular pathways

(76) Inventors: Marvin A. Heuer, Mississauga (CA); Shan Chaudhuri, Mississauga (CA); Ken Clement, Mississauga (CA)

Correspondence Address: KENYON & KENYON LLP, ONE BROADWAY, NEW YORK, NY, 10004, US

(21) Appl. No.: 11/595,170

(22) Filed: Nov. 8, 2006

Related U.S. Application Data
(60) Provisional application No. 60/735,057, filed on Nov. 8, 2005.
(60) Provisional application No. 60/776,325, filed on Feb. 23, 2006.

(51) Int. Cl. A61K 31/405; A61K 31/401; A61K 31/198; A61K 31/385; A61K 31/19; A61K 31/185

(52) U.S. Cl. 514/419; 514/423; 514/557; 514/561; 514/553; 514/565; 514/566; 514/567; 514/440

(57) ABSTRACT

Compositions and methods relating to the use of cationic and monobasic amino acids combined with ketoacids for the purposes of regulating molecular signals to control anabolic and anti-catabolic activity in skeletal muscular development are disclosed. The present invention is designed to influence the genetic control system for muscle protein synthesis to stimulate muscle growth, increase muscle mass, decrease muscle catabolism, decrease recovery time and to treat muscle wasting and/or degeneration. Said compositions are to be administered to a subject one to four times daily.

### RELATED APPLICATIONS

The application is related to and claims benefit of priority to U.S. Provisional Patent Application Ser. No. 60/735,057 entitled “Supplemental Dietary Composition for Turning on Anabolic Switches in Muscle, Stimulating and/or Optimizing Protein Synthesis, and/or Potently Signaling Muscle Building and/or Growth,” filed Nov. 8, 2005, the disclosure of which is hereby fully incorporated by reference. Benefit of priority of is also claimed to the applicant's co-pending U.S. Provisional Patent Application Ser. No. 60/776,325 entitled “Compositions and method for increasing bioavailability of compositions for performance improvement”, file Feb. 23, 2006, the disclosure of which is hereby fully incorporated by reference.

### FIELD OF THE INVENTION

The present invention relates to a supplemental dietary composition that comprises a ketoacid in combination with one or more cationic or monobasic amino acids. In addition, the present invention relates to a method for, e.g. turning on anabolic switches in muscle, stimulating and optimizing protein synthesis, and/or potently signaling muscle building and/or growth and reducing nitrogen load. In addition, the present invention relates to a method of manufacturing the supplemental dietary composition.

### SUMMARY OF THE INVENTION

The present invention provides a method of regulating molecular signals to control anabolic and anti-catabolic activity in skeletal muscle via a combination of cationic or monobasic amino acids and ketoacids. For example, the present invention may provide, by the consumption of a supplemental dietary composition as set forth herein, a method for stimulating muscle growth, increasing muscle mass, decreasing muscle catabolism and associated muscle and weight loss, increasing performance, decreasing recovery time, improving body composition, treating muscle wasting and/or degeneration and/or providing a beneficial effect by influencing the genetic control system for global protein synthesis. Most specifically, the present invention provides a composition and method for turning on anabolic switches in muscle, stimulating and optimizing protein synthesis, as well as potently signaling muscle building and/or growth. Advantageously, consumption of the supplemental dietary composition is combined with a calorie limited diet and a regular program of exercise.

The present invention additionally provides a dietary supplement that is comprised of a ketoacid in combination with one or more cationic or monobasic amino acids. Furthermore, fine-milled amino acids and ketoacids may used alone or in combination to comprise the present invention. Fine-milled particles having an average size from about 2 to about 50 microns may be used to increase the bioavailability of the components comprising the invention.

In addition, the present invention relates to a method of manufacturing a supplemental dietary composition that may regulate molecular signals to control anabolic and anti-catabolic activity in skeletal muscle, and in doing so, may stimulate muscle growth, increase muscle mass, decrease muscle catabolism and associated muscle and weight loss, increase performance, decrease recovery time, improve body composition, treat muscle wasting and/or degeneration and/or provide a beneficial effect by influencing the genetic control system for global protein synthesis. In various embodiments, the method of manufacturing a supplemental dietary composition includes the step of e.g., mixing a ketoacid with one or more than one cationic or monobasic amino acid selected form the group comprised of glycine, creatine, alanine, valine, leucine, isoleucine, methionine, proline, phenylalanine, tryptophan, serine, threonine, asparagine, glutamine, tyrosine, cysteine, glutamic acid aspartic acid and taurine.

### BRIEF DESCRIPTION OF DRAWINGS

FIG. 1 Is a schematic diagram of the activation of the mTOR intracellular pathway leading to muscle protein synthesis.

### DETAILED DESCRIPTION OF THE INVENTION

U.S. Pat. No. 6,100,287 describes a method for enhancing muscle performance recovery from fatigue wherein the method includes administering a composition of a cationic or dibasic amino acid and a ketoacid. The invention is further detailed in a specific embodiment comprising glycine, L-Arginine monohydrochloride salt of alpha-ketoisocaproic acid calcium.

Conventionally, amino acids have been seen as precursors of protein synthesis. It has recently been demonstrated that key amino acids are able to regulate mRNA translation and can be used to directly activate protein. synthesis for the purposes of muscle growth and development (Yoshizawa F. Regulation of protein synthesis by branched-chain amino acids in vivo. Biochem Biophys Res Commun. Jan. 9, 2004;313(2):417-22. Review). More specifically, growth factors such as insulin and monobasic or cationic amino acids such as e.g., glycine, creatine, alanine, valine, leucine, isoleucine, methionine, proline, phenylalanine, tryptophan, serine, threonine, asparagine, glutamine, tyrosine, cysteine, glutamic acid aspartic acid and taurine, are involved in the key intracellular pathways regulating muscle protein synthesis. Both insulin and the aforementioned amino acids directly modify critical points (Fingar D C, Richardson C J, Tee A R, Cheatham L, Tsou C, Blenis J. mTOR controls cell cycle progression through its cell growth effectors S6K1 and 4E-BP1/eukaryotic translation initiation factor 4E. Mol Cell Biol. January 2004;24(1):200-16.) in muscle development to activate the protein kinase mTOR (mammalian target of rapamycin), a site of integration of signals that stimulates muscle protein synthesis, cell growth and size as well as progression into the cell cycle (Fingar D C, Richardson C J, Tee A R, Cheatham L, Tsou C, Blenis J. mTOR controls cell cycle progression through its cell growth effectors S6K1 and 4E-BP1/eukaryotic translation initiation factor 4E. Mol Cell Biol. January 2004;24(1):200-16.).

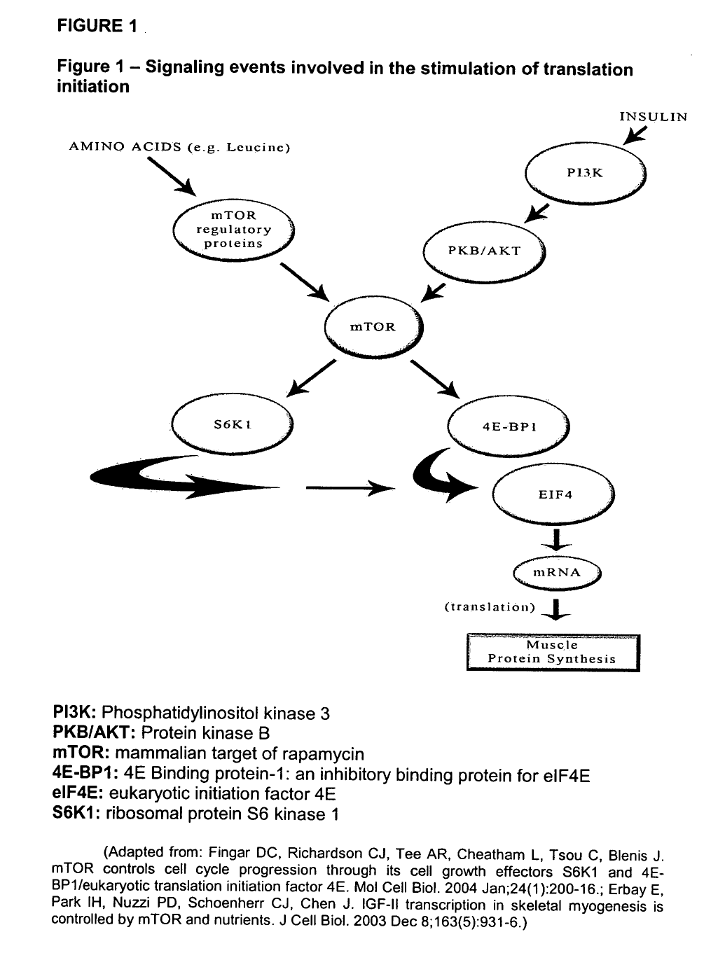

Leucine as well as other amino acids are key components in the aforementioned formula. Notably, they have been found and implicated in stimulating muscle protein synthesis, with Leucine being the most potent branched-chain amino acid for stimulating muscle protein synthesis (Yoshizawa F. Regulation of protein synthesis by branched-chain amino acids in vivo. Biochem Biophys Res Commun. Jan. 9, 2004;313(2):417-22. Review). There are also effects mediated via a rapamycin independent mechanism. Using e.g., glycine, creatine, alanine, valine, leucine, isoleucine, methionine, proline, phenylalanine, tryptophan, serine, threonine, asparagine, glutamine, tyrosine, cysteine, glutamic acid aspartic acid and taurine or other derivatives or bound forms of these monobasic amino acids, as set forth in additional detail below, with or without the addition of simple sugars, to elicit an insulin spike, this protein synthesis pathway, when triggered, can stimulate the initiation of mRNA translation for muscle growth (Cuthbertson D, Smith K, Babraj J, Leese G, Waddell T, Atherton P, Wackerhage H, Taylor P M, Rennie M J. Anabolic signaling deficits underlie amino acid resistance of wasting, aging muscle. FASEB J. March 2005;19(3):422-4. Epub Dec. 13, 2004; Lang C H, Frost R A. Endotoxin disrupts the leucine-signaling pathway involving phosphorylation of mTOR, 4E-BP1, and S6K1 in skeletal muscle. J Cell Physiol. April 2005;203(1):144-55.). FIG. 1 is diagram which illustrates the signaling events which are involved in the stimulation of translation initiation. More specifically, FIG. 1 illustrates how both amino acids and insulin can activate mTOR to trigger the phosphorylation of 4E-BP1 and S6k1 (and other key proteins, i.e. p70^{S6K}), leading to the release of eIF4E (enhancing association of eIF4E with eIF4G) and ultimately leading to an increase in protein synthesis and inhibition of protein catabolism.

According to the present invention, ketoacids, in combination with monobasic amino acids may promote protein synthesis and inhibit the degradation of skeletal muscle by influencing the net protein balance controlled at a genetic level. Generally, the present invention, in accordance with various embodiments thereof, provides a novel dietary supplement that comprises of one or more than one monobasic amino acid(s), e.g., glycine, creatine, alanine, valine, leucine, isoleucine, methionine, proline, phenylalanine, tryptophan, serine, threonine, asparagine, glutamine, tyrosine, cysteine, glutamic acid, aspartic acid and taurine in conjunction with ketoacids, with or without simple carbohydrates, to directly and in directly control key molecular pathways involving AKT/PKB and mTOR to influence gene expression in order to stimulate mRNA translation for skeletal muscle growth (Raught B, Gingras A C. eIF4E activity is regulated at multiple levels. Int J Biochem Cell Biol. January 1999;31(1):43-57. Review.).

As used herein, ketoacids may include, for example, any one of the following, but not limited to: alpha-ketoisocaproic acid (KIC), alpha-ketoglutaric acid, alpha-ketoisovaleric acid, alpha-ketobetmethylvaleric acid, pyruvic acid, and salts thereof. When amino acids are metabolized to be used a cellular fuel, ammonia results as a by-product (Groff J L, Gropper S S. Advanced Nutrition and Human Metabolism, 3rd Edition. Wadsworth Thomson Learning. Scarborough, Ontario. 19 pg 187-188). In addition to arising from the deanimation of amino acids, ammonia also arises from the deanimation of adenosine triphosphate to inosine monophosphate in the purine nucleotide cycle. Ketoacids have been shown to re-animate amino acids as a means to treat muscle wasting in acutely traumatized as well as critically ill patients. Moreover, ketoacids have been shown to reduce nitrogen load by using free ammonia in serum to re-animate ketoacids (Harper A E, Miller R H, Block K P. Branched-chain amino acid metabolism. Annu Rev Nutr. 1984;4:409-54. Review). Furthermore, ketoacid/amino acid complexes have been shown to enhance injury repair, improve trauma recovery time, enhance injury repair reduce serum ammonia (Chesley A, MacDougall J D, Tarnopolsky M A, Atkinson S A, Smith K. Changes in human muscle protein synthesis after resistance exercise. J Appl Physiol. October 1992;73(4):1383-8).

For example, the present invention, according to various embodiments thereof, provides a dietary supplement comprising a ketoacid in combination with one or more of a cationic or monobasic amino acid selected from the group comprised of glycine, creatine, alanine, valine, leucine, isoleucine, methionine, proline, phenylalanine, tryptophan, serine, threonine, asparagine, glutamine, tyrosine, cysteine, glutamic acid, aspartic acid and taurine. Furthermore, fine-milled amino acids and ketoacids may used alone or in combination. Moreover, additional ingredients may be included as excipients, but not limited to, alone or in combination those selected from the group consisting of hydroxypropyl cellulose, microcrystalline cellulose, croscarmellose sodium, calcium carbonate, vegetable stearine, magnesium stearate, silica, magnesium silicate, Leucoat™ (polyvinyl alcohol, polyethylene glycol, talc, titanium dioxide, riboflavin, colorings, hydroxypropyl cellulose, soy lecithin, polysorbate 80), hydroxypropyl methylcellulose, and sweeteners in a caplet form. In a powdered beverage form, excipients may include, but not limited to, alone or in combination, citric acid, prosweet bitterness mask, bitterness mask, orange flavor, pineapple flavor, veltol ultra, anti-foam fluid spray, sweeteners, sucralose, and colorings.

In an embodiment of the present invention, which is set forth in greater detail in Example 1 below, the supplemental dietary composition may include glycine in combination with a ketoacid, and in particular may include glycine-KIC. For example, in the embodiment set forth in Example 1, the supplemental dietary composition includes glycine-KIC in an amount ranging from about 5.000 grams to about 10.000 grams per serving.

In an embodiment of the present invention, which is set forth in greater detail in Example 2 below, the supplemental dietary composition may include alanine in combination with a ketoacid, and in particular may include alanine-KIC. For example, in the embodiment set forth in Example 2, the supplemental dietary composition includes alanine-KIC in an amount ranging from about 5.000 grams to about 10.000 grams per serving.

In an embodiment of the present invention, which is set forth in greater detail in Example 3 below, the supplemental dietary composition may include valine in combination with a ketoacid, and in particular may include valine-KIC. For example, in the embodiment set forth in Example 3, the supplemental dietary composition includes valine-KIC in an amount ranging from about 5.000 grams to about 10.000 grams per serving.

In an embodiment of the present invention, which is set forth in greater detail in Example 4 below, the supplemental dietary composition may include leucine in combination with a ketoacid, and in particular may include leucine-KIC. For example, in the embodiment set forth in Example 4, the supplemental dietary composition includes leucine-KIC in an amount ranging from about 5.000 grams to about 10.000 grams per serving.

In an embodiment of the present invention, which is set forth in greater detail in Example 5 below, the supplemental dietary composition may include isoleucine in combination with a ketoacid, and in particular may include isoleucine-KIC. For example, in the embodiment set forth in Example 5, the supplemental dietary composition includes isoleucine-KIC in an amount ranging from about 5.000 grams to about 10.000 grams per serving.

In an embodiment of the present invention, which is set forth in greater detail in Example 6 below, the supplemental dietary composition may include methionine in combination with a ketoacid, and in particular may include methionine-KIC. For example, in the embodiment set forth in Example 6, the supplemental dietary composition includes methionine-KIC in an amount ranging from about 5.000 grams to about 10.000 grams per serving.

In an embodiment of the present invention, which is set forth in greater detail in Example 7 below, the supplemental dietary composition may include proline in combination with a ketoacid, and in particular may include proline-KIC. For example, in the embodiment set forth in Example 7, the supplemental dietary composition includes proline-KIC in an amount ranging from about 5.000 grams to about 10.000 grams per serving.

In an embodiment of the present invention, which is set forth in greater detail in Example 8 below, the supplemental dietary composition may include phenylalanine in combination with a ketoacid, and in particular may include phenylalanine-KIC. For example, in the embodiment set forth in Example 8, the supplemental dietary composition includes phenylalanine-KIC in an amount ranging from about 5.000 grams to about 10.000 grams per serving.

In an embodiment of the present invention, which is set forth in greater detail in Example 9 below, the supplemental dietary composition may include tryptophan in combination with a ketoacid, and in particular may include tryptophan-KIC. For example, in the embodiment set forth in Example 9, the supplemental dietary composition includes tryptophan-KIC in an amount ranging from about 5.000 grams to about 10.000 grams per serving.

In an embodiment of the present invention, which is set forth in greater detail in Example 10 below, the supplemental dietary composition may include serine in combination with a ketoacid, and in particular may include serine-KIC. For example, in the embodiment set forth in Example 10, the supplemental dietary composition includes serine-KIC in an amount ranging from about 5.000 grams to about 10.000 grams per serving.

In an embodiment of the present invention, which is set forth in greater detail in Example 11 below, the supplemental dietary composition may include threonine in combination with a ketoacid, and in particular may include threonine-KIC. For example, in the embodiment set forth in Example 11, the supplemental dietary composition includes threonine-KIC in an amount ranging from about 5.000 grams to about 10.000 grams per serving.

In an embodiment of the present invention, which is set forth in greater detail in Example 12 below, the supplemental dietary composition may include asparagine in combination with a ketoacid, and in particular may include asparagine-KIC. For example, in the embodiment set forth in Example 12, the supplemental dietary composition includes asparagine-KIC in an amount ranging from about 5.000 grams to about 10.000 grams per serving.

In an embodiment of the present invention, which is set forth in greater detail in Example 13 below, the supplemental dietary composition may include glutamine in combination with a ketoacid, and in particular may include glutamine-KIC. For example, in the embodiment set forth in Example 13, the supplemental dietary composition includes glutamine-KIC in an amount ranging from about 5.000 grams to about 10.000 grams per serving.

In an embodiment of the present invention, which is set forth in greater detail in Example 14 below, the supplemental dietary composition may include tyrosine in combination with a ketoacid, and in particular may include tyrosine-KIC. For example, in the embodiment set forth in Example 14, the supplemental dietary composition includes tyrosine-KIC in an amount ranging from about 5.000 grams to about 10.000 grams per serving.

In an embodiment of the present invention, which is set forth in greater detail in Example 15 below, the supplemental dietary composition may include cysteine in combination with a ketoacid, and in particular may include cysteine-KIC. For example, in the embodiment set forth in Example 15, the supplemental dietary composition includes cysteine-KIC in an amount ranging from about 5.000 grams to about 10.000 grams per serving.

In an embodiment of the present invention, which is set forth in greater detail in Example 16 below, the supplemental dietary composition may include glutamic acid in combination with a ketoacid, and in particular may include glutamic acid-KIC. For example, in the embodiment set forth in Example 16, the supplemental dietary composition includes glutamic-KIC in an amount ranging from about 5.000 grams to about 10.000 grams per serving.

In an embodiment of the present invention, which is set forth in greater detail in Example 17 below, the supplemental dietary composition may include aspartic acid in combination with a ketoacid, and in particular may include aspartic acid-KIC. For example, in the embodiment set forth in Example 17, the supplemental dietary composition includes aspartic acid in an amount ranging from about 5.000 grams to about 10.000 grams per serving.

In an embodiment of the present invention, which is set forth in greater detail in Example 18 below, the supplemental dietary composition may include taurine in combination with a ketoacid, and in particular may include taurine-KIC. For example, in the embodiment set forth in Example 18, the supplemental dietary composition includes taurine-KIC in an amount ranging from about 5.000 grams to about 10.000 grams per serving.

In an embodiment of the present invention which is set for in greater detail in Example 19 below, the supplemental dietary composition may include a ketoacid in combination with Leucine or other, but not limited to, of the aforementioned amino acids, and in particular may include Leucine-ketoisovalerate. For example, in the embodiment set forth in Example 19, the supplement dietary composition includes Leucine-ketoisovalerate and/or salts thereof in an amount ranging from about 5.000 grams to about 10.000 grams per serving.

In an embodiment of the present invention which is set for in greater detail in Example 20 below, the supplemental dietary composition may include a ketoacid in combination with Leucine or other, but not limited to, of the aforementioned amino acids, and in particular may include Leucine-ketobetamethylvalerate. For example, in the embodiment set forth in Example 20, the supplement dietary composition includes Leucine-ketobetamethylvalerate and/or salts thereof in an amount ranging from about 5.000 grams to about 10.000 grams per serving.

In an embodiment of the present invention which is set for in greater detail in Example 21 below, the supplemental dietary composition may include a ketoacid in combination with Leucine or other, but not limited to, of the aforementioned amino acids, and in particular may include Leucine-pyruvate. For example, in the embodiment set forth in Example 21, the supplement dietary composition includes Leucine-pyruvate and/or salts thereof in an amount ranging from about 5.000 grams to about 10.000 grams per serving.

In an embodiment of the present invention which is set for in greater detail in Example 22 below, the supplemental dietary composition may include a ketoacid in combination with Leucine or other, but not limited to, of the aforementioned amino acids, and in particular may include Leucine-α-ketoglutarate. For example, in the embodiment set forth in Example 22, the supplement dietary composition includes Leucine-α-ketoglutarate and/or salts thereof in an amount ranging from about 5.000 grams to about 10.000 grams per serving.

In an embodiment of the present invention which is set for in greater detail in Example 23 below, the supplemental dietary composition may include a ketoacid in combination with Glutamate or other, but not limited to, of the aforementioned amino acids, and in particular may include Glutamine-α-ketoglutarate. For example, in the embodiment set forth in Example 23, the supplement dietary composition includes Glutamine-α-ketoglutarate and/or salts thereof in an amount ranging from about 5.000 grams to about 10.000 grams per serving.

In an embodiment of the present invention, which is set forth in greater detail in Example 24 below, the supplemental dietary composition may include an amino acid, such as Leucine, but not limited to, in combination, but not limited to a ketoacid such as calcium-KIC. For example, in the embodiment set forth in Example 24, the supplemental dietary composition includes Leucine plus calcium-KIC with addition of maltodextrin in an amount ranging from about 5.000 grams to about 10.000 grams per serving.

In an embodiment of the present invention, which is set forth in greater detail in Example 25 below, the supplemental dietary composition may include an amino acid, such as Creatine, but not limited to, in combination, but not limited to a ketoacid such as calcium-KIC and Alpha Lipoic Acid. For example, in the embodiment set forth in Example 25, the supplemental dietary composition includes Creatine plus calcium-KIC with addition of Alpha Lipoic Acid in an amount ranging from about 5.000 grams to about 10.000 grams per serving.

In an embodiment of the present invention which is set for in greater detail in Example 26 below, the supplemental dietary composition may include a ketoacid in combination with Creatine or other, but not limited to, of the aforementioned amino acids, and in particular may include creatine-α-ketoglutarate. For example, in the embodiment set forth in Example 26, the supplement dietary composition includes creatine-α-ketoglutarate and/or salts thereof in an amount ranging from about 5.000 grams to about 10.000 grams per serving.

In an embodiment of the present invention which is set for in greater detail in Example 27 below, the supplemental dietary composition may include a ketoacid in combination with Creatine or other, but not limited to, of the aforementioned amino acids, and in particular may include creatine-ketoisocaproic acid. For example, in the embodiment set forth in Example 27, the supplement dietary composition includes creatine-ketoisocaproic acid and/or salts thereof in an amount ranging from about 5.000 grams to about 10.000 grams per serving.

The present invention may also provide a method of regulating molecular signals to control anabolic and anti-catabolic activity in skeletal muscle via the combination of monobasic amino acids and ketoacids. For example, the present invention may provide, by the consumption of a dietary supplement as set forth herein, a method for stimulating muscle growth, increasing muscle mass, decreasing muscle catabolism and associated muscle and weight loss, increasing performance, decreasing recovery time, improving body composition, treating muscle wasting and/or degeneration and/or providing a beneficial effect by influencing the genetic control system for global protein synthesis. Specifically, the present invention provides a composition and method for turning on anabolic switches in muscle, stimulating and optimizing protein synthesis, as well as potently signaling muscle building and/or growth. Advantageously, consumption of the dietary supplement is combined with a calorie limited diet and a regular program of exercise.

As set forth above, the use of, e.g., glycine, creatine, alanine, valine, leucine, isoleucine, methionine, proline, phenylalanine, tryptophan, serine, threonine, asparagine, glutamine, tyrosine, cysteine, glutamic acid aspartic acid and taurine, alone or in combination, in conjunction with ketoacids as set forth in the example embodiments listed above, may provide various effects or benefits. For example, supplemental dietary compositions may perform, provide, or enable one or more of the following: genetic manipulation for advanced muscle growth; genetically manipulates molecular mechanism for muscle growth; genetically enhance muscle growth; gene powered muscle building; genetically induce muscle growth; genetically stimulate muscle building; genetic muscle promoter; regulate skeletal muscle growth; stimulate muscle development; mediate skeletal muscle homeostasis; regulate muscle's genetic potential; genetic muscle growth stimulator for genetically optimized muscle growth; stimulate gene expression for muscle growth; directly promote muscle protein synthesis; turn on muscle promoting pathways; stimulate muscle growth; stimulate mRNA translation for muscle growth; initiate mRNA translation for muscle growth; accelerate muscle protein synthesis; activate mTOR expression to turn on protein synthesis; ketoacids assist in reducing nitrogen load; intracellular regulation of protein building; optimizes muscle accretion; regulate signaling mechanisms to promote anabolism; turns on anabolic switches; switch off catabolism switches; regulate signaling mechanisms to inhibit catabolism; phophorylate key proteins involved in regulating muscle growth; reach your full genetic potential; reach maximum protein synthesis rate; breakthrough your genetic barriers; optimize muscle growth; muscle growth activator; direct muscle growth stimulator; potent anabolism promoter; intense anabolic signaling agent; push you past your genetic potential; directly turn on anabolic switches in muscles; potently enhance muscle growth; directly activate muscle building pathways; regulates anabolic mechanisms in muscle; most powerful anabolic nutrient/molecule; optimize muscle protein synthesis stimulation; escalate anabolic signaling at the molecular level; intense protein synthesis stimulation; advanced anabolic nutrient signaling; genetically induce muscle hypertrophy; genetically enhance muscle strength; and genetic control over muscle growth.

According to various embodiments of the present invention, the dietary supplement may be consumed in any form. For instance, the dosage form of the supplemental dietary composition may be provided as e.g., a powder beverage mix, a liquid beverage, a ready-to-eat bar or drink product, a capsule, a tablet, a caplet, or as a dietary gel. The most preferred dosage forms are caplets or a powdered beverage mix. The dietary supplement may be consumed any number of times per day to in order to obtain any one of the benefits set forth above. As set forth herein, the dietary supplement as indicated in examples of the present invention, are preferably consumed one to four times per day in order to obtain any one of the benefits outlined in this document.

Furthermore, the dosage form of the dietary supplement may be provided in accordance with customary processing techniques for herbal and/or dietary supplements in any of the aforementioned forms. Moreover, the supplemental dietary composition set forth in the example embodiments listed herein may contain any appropriate number and type of excipients as outline in the Examples and as known in the art.

In addition, the present invention comprises a method of manufacturing for a supplemental dietary composition that may act to turn on anabolic switches in muscle, stimulating and/or optimizing protein synthesis, and/or potently signaling muscle building and/or growth. For example, the method of manufacturing a dietary supplement may include the step of mixing a ketoacid with one or more than one monobasic or cationic amino acid selected from the group consisting of glycine, creatine, alanine, valine, leucine, isoleucine, methionine, proline, phenylalanine, tryptophan, serine, threonine, asparagine, glutamine, tyrosine, cysteine, glutamic acid, aspartic acid and/or taurine. The method of manufacturing the dietary supplement may also include the step checking for uniformity and/or homogeneity. Furthermore, the method of manufacturing the dietary supplement may include the step of aliquoting the mixture into a serving for, e.g., compression into a caplet or powdered beverage mix. As set forth above, the dosage form of the diet supplement, in accordance with the example embodiments set forth below, may be provided in accordance with customary processing techniques for herbal and/or dietary supplements, wherein the active ingredients are suitably processed into a desired form. In accordance with one embodiment of the present invention, one or more ingredients of the diet supplement are processed so as to form fine-milled particles. For instance, in one embodiment, one or more ingredients of the supplemental dietary composition is processed by a large-scale dry milling technique that produces fine particles, preferably known as fine-milled particles. The use of dry milling techniques, in combination with excipients and polymers, to form fine-milled particles has been shown to improve flow and dispersability, stability, resistance to moisture, bioavailability, and dissolution/release properties. Formulations benefit by containing fine-milled particles for the purpose of providing the one or more ingredients in particle sizes that optimize one or more of the flow and dispersability, stability, resistance to moisture, bioavailability, and dissolution/release properties of the one or more ingredients in a diet supplement. In vitro tests designed to simulate the environment of stomach were preformed to test the dissolution rate of fine-milled particle tablets versus non-fine-milled. These test showed that in tablets produced from fine-milled particles the time to 100% dissolution was approximately 15 minutes. In the case of non-fine-milled particle compositions, only 90% dissolution was observed after 120 minutes. In a preferred embodiment, the supplemental dietary composition contains fine-milled particles having and average size between about 50 nm and about 2 nm.

U.S. Provisional Patent Application 60/776,325 discloses a method for improving the absorption, palatability, taste, texture, and bioavailability of compounds by increasing the solubility of compounds in proprietary formulations for the purposes of enhancing or improving muscle size, growth and/or recovery time and/or weight loss. The increased bioavailability of the compound or ingredients is achieved by reducing the particle size via “fine-milling” thereby increasing the surface area-to-volume ratio each particle, thus increasing the rate of dissolution. The compositions and methods disclosed promote increased bioavailability by increasing the total surface area of poorly soluble particles, thereby increasing the rate of absorption.

As used herein the, term “fine-milled” and/or “fine-milling” refers to the process of micronization. Micronization is a mechanical process that involves the application of force to a particle, thereby resulting in a reduction in the size of the particle. The force, in the case of micronization may be applied in any manner such as, e.g., the collision of particles at high rates of speed, grinding, or by an air-jet micronizer. In a preferred embodiment, fine-milled particles are obtained by jet-milling with nitrogen and compressed air.

As used herein, term “particle size” refers to the diameter of the particle. The term “average particle size” means that at least 50% of the particles in a sample will have the specified particle size. Preferably, at least 80% of the particles in a sample will have the specified particle size, and more preferably, at least 90% of the particles in a given sample will have the specified particle size.

The size of a particle can be determined any of the method known within the art. Methods for particle size determination which may be employed are for example, e.g., sieves, sedimentation, electrozone sensing (Coulter counter), microscopy, and/or Low Angle Laser Light Scattering. The preferred methods for the particle size determination of the present invention are the methods which are most commonly used in the pharmaceutical industry, such as laser diffraction, e.g., via light scattering Coulter Delsa 440SX.

The fine-milling process may be employed in the processing of one or more of the ingredients of the present invention in the dosage forms of tablets, e.g., immediate-release film coated, modified-release and fast-dissolving; capsules, e.g., immediate-release and modified-release; liquid dispersions; powders; drink mixes, etc.

By activating signal transduction pathways which are both mTOR dependant and independent, the present invention provides a novel method to ensure the anabolic protein machinery with in the cell is operating in favorable manner to promote an anabolic environment within muscles to assist in optimizing protein synthesis. The present invention may provide an advantage over conventional products that purport to stimulate protein synthesis but, lack, or include in insufficient quantities, the correct signaling promoting nutritive agents, such as branch-chain as well as monobasic amino acids to ensure proper RNA translation initiation for muscle building. Moreover, the present invention may provide a method of reducing nitrogen load by reanimating ketoacids.

Although the following examples illustrate the practice of the present invention in several embodiments, the examples should not be taken as limiting the scope of the invention. Other embodiments will be apparent to those skilled in the art from consideration of the specification of the following example.

### EXAMPLES

### Example 1

A dietary supplement comprising Glycine-alpha-ketoisocaproic acid in amounts from about 5 g to about 10 g per serving is prepared for consumption one to four times per day per individual. The dosage form provided comprises either a caplet or powder beverage mix.

### Example 2

A dietary supplement comprising Alanine-alpha-ketoisocaproic acid in amounts from about 5 g to about 10 g per serving is prepared for consumption one to four times per day per individual. The dosage form provided comprises either a caplet or powder beverage mix.

### Example 3

A dietary supplement comprising Valine-alpha-ketoisocaproic acid in amounts from about 5 g to about 10 g per serving is prepared for consumption one to four times per day per individual. The dosage form provided comprises either a caplet or powder beverage mix.

### Example 4

A dietary supplement comprising Leucine-alpha-ketoisocaproic acid in amounts from about 5 g to about 10 g per serving is prepared for consumption one to four times per day per individual. The dosage form provided comprises either a caplet or powder beverage mix.

### Example 5

A dietary supplement comprising Isoleucine-alpha-ketoisocaproic acid in amounts from about 5 g to about 10 g per serving is prepared for consumption one to four times per day per individual. The dosage form provided comprises either a caplet or powder beverage mix.

### Example 6

A dietary supplement comprising Methionine-alpha-ketoisocaproic acid in amounts from about 5 g to about 10 g per serving is prepared for consumption one to four times per day per individual. The dosage form provided comprises either a caplet or powder beverage mix.

### Example 7

A dietary supplement comprising Proline-alpha-ketoisocaproic acid in amounts from about 5 g to about 10 g per serving is prepared for consumption one to four times per day per individual. The dosage form provided comprises either a caplet or powder beverage mix.

### Example 8

A dietary supplement comprising Phenylalanine-alpha-ketoisocaproic acid in amounts from about 5 g to about 10 g per serving is prepared for consumption one to four times per day per individual. The dosage form provided comprises either a caplet or powder beverage mix.

### Example 9

A dietary supplement comprising Tryptophan-alpha-ketoisocaproic acid in amounts from about 5 g to about 10 g per serving is prepared for consumption one to four times per day per individual. The dosage form provided comprises either a caplet or powder beverage mix.

### Example 10

A dietary supplement comprising Serine-alpha-ketoisocaproic acid in amounts from about 5 g to about 10 g per serving is prepared for consumption one to four times per day per individual. The dosage form provided comprises either a caplet or powder beverage mix.

### Example 11

A dietary supplement comprising Threonine-alpha-ketoisocaproic acid in amounts from about 5 g to about 10 g per serving is prepared for consumption one to four times per day per individual. The dosage form provided comprises either a caplet or powder beverage mix.

### Example 12

A dietary supplement comprising Asparagine-alpha-ketoisocaproic acid in amounts from about 5 g to about 10 g per serving is prepared for consumption one to four times per day per individual. The dosage form provided comprises either a caplet or powder beverage mix.

### Example 13

A dietary supplement comprising Glutamine-alpha-ketoisocaproic acid in amounts from about 5 g to about 10 g per serving is prepared for consumption one to four times per day per individual. The dosage form provided comprises either a caplet or powder beverage mix.

### Example 14

A dietary supplement comprising Tyrosine-alpha-ketoisocaproic acid in amounts from about 5 g to about 10 g per serving is prepared for consumption one to four times per day per individual. The dosage form provided comprises either a caplet or powder beverage mix.

### Example 15

A dietary supplement comprising Cysteine-alpha-ketoisocaproic acid in amounts from about 5 g to about 10 g per serving is prepared for consumption one to four times per day per individual. The dosage form provided comprises either a caplet or powder beverage mix.

### Example 16

A dietary supplement comprising Glutamic acid-alpha-ketoisocaproic acid in amounts from about 5 g to about 10 g per serving is prepared for consumption one to four times per day per individual. The dosage form provided comprises either a caplet or powder beverage mix.

### Example 17

A dietary supplement comprising Aspartic acid-alpha-ketoisocaproic acid in amounts from about 5 g to about 10 g per serving is prepared for consumption one to four times per day per individual. The dosage form provided comprises either a caplet or powder beverage mix.

### Example 18

A dietary supplement comprising Taurine-alpha-ketoisocaproic acid in amounts from about 5 g to about 10 g per serving is prepared for consumption one to four times per day per individual. The dosage form provided comprises either a caplet or powder beverage mix.

### Example 19

A dietary supplement comprising Leucine-ketoisovaleric acid in amounts from about 5 g to about 10 g per serving is prepared for consumption one to four times per day per individual. The dosage form provided comprises either a caplet or powder beverage mix.

### Example 20

A dietary supplement comprising Leucine-ketobetamethylvalerate in amounts from about 5 g to about 10 g per serving is prepared for consumption one to four times per day per individual. The dosage form provided comprises either a caplet or powder beverage mix.

### Example 21

A dietary supplement comprising Leucine-pyruvate in amounts from about 5 g to about 10 g per serving is prepared for consumption one to four times per day per individual. The dosage form provided comprises either a caplet or powder beverage mix.

### Example 22

A dietary supplement comprising Leucine-alpha-ketoglutarate in amounts from about 5 g to about 10 g per serving is prepared for consumption one to four times per day per individual. The dosage form provided comprises either a caplet or powder beverage mix.

### Example 23

A dietary supplement comprising Glutamine-alpha-ketoglutarate in amounts from about 5 g to about 10 g per serving is prepared for consumption one to four times per day per individual. The dosage form provided comprises either a caplet or powder beverage mix.

### Example 24

A dietary supplement comprising fine-milled Leucine, Maltodextrin and Calcium-ketoisocaproic acid in a total amount from about 5 g to about 10 g per serving is prepared for consumption one to four times per day per individual. The dosage form provided comprises either a caplet or powder beverage mix.

### Example 25

A dietary supplement comprising fine-milled Creatine, Alpha Lipoic Acid and Calcium-ketoisocaproic acid in a total amount from about 5 g to about 10 g per serving is prepared for consumption one to four times per day per individual. The dosage form provided comprises either a caplet or powder beverage mix.

### Example 26

A dietary supplement comprising creatine-a-ketoglutarate in amounts from about 5 g to about 10 g per serving is prepared for consumption one to four times per day per individual. The dosage form provided comprises either a caplet or powder beverage mix.

### Example 27

A dietary supplement comprising creatine-ketoisocaproate in amounts from about 5 g to about 10 g per serving is prepared for consumption one to four times per day per individual. The dosage form provided comprises either a caplet or powder beverage mix.

What is claimed:

1. A composition comprising a monobasic amino acid and at least one ketoacid or salt thereof.

2. The composition of claim 1 wherein the monobasic amino acid is selected from the group consisting of glycine, creatine, alanine, valine, leucine, isoleucine, methionine, proline, phenylalanine, tryptophan, serine, threonine, asparagine, glutamine, tyrosine, cysteine, glutamic acid aspartic acid and taurine.

3. The composition of claim 1 wherein the ketoacids are selected from the group consisting of alpha-ketoisocaproic acid, alpha-ketoglutaric acid, alpha-ketoisovaleric acid, alpha-ketobetamethylvaleric acid, pyruvic acid, and salts thereof.

4. The composition of claim 1 wherein the alpha-ketoisocaproic calcium salt comprises from about 0.01 g to about 0.06 g of a serving.

5. The composition of claim 4 wherein fine-milled Leucine comprises from about 6.50 g to about 7.50 g of a serving.

6. The composition of claim 4 further comprising from about 2 g to about 3 of creatine and from about 0.001 g to about 0.02 g of alpha lipoic acid.

7. The composition of claim 1, comprising Glycine and alpha-ketoisocaproic acid or salts thereof in various amounts to a weight per serving of from about 5 g to about 10 g.

8. The composition of claim 1, comprising Alanine and alpha-ketoisocaproic acid or salts thereof in various amounts to a weight per serving of from about 5 g to about 10 g.

9. The composition of claim 1, comprising Valine and alpha-ketoisocaproic acid or salts thereof in various amounts to a weight per serving of from about 5 g to about 10 g.

10. The composition of claim 1, comprising Leucine and alpha-ketoisocaproic acid or salts thereof in various amounts to a weight per serving of from about 5 g to about 10 g.

11. The composition of claim 1, comprising Isoleucine and alpha-ketoisocaproic acid or salts thereof in various amounts to a weight per serving of from about 5 g to about 10 g.

12. The composition of claim 1, comprising Methionine and alpha-ketoisocaproic acid or salts thereof in various amounts to a weight per serving of from about 5 g to about log.

13. The composition of claim 1, comprising Proline and alpha-ketoisocaproic acid or salts thereof in various amounts to a weight per serving of from about 5 g to about 10 g.

14. The composition of claim 1, comprising Phenylalanine and alpha-ketoisocaproic acid or salts thereof in various amounts to a weight per serving of from about 5 g to about 10 g.

15. The composition of claim 1, comprising Tryptophan and alpha-ketoisocaproic acid or salts thereof in various amounts to a weight per serving of from about 5 g to about 10 g.

16. The composition of claim 1, comprising Serine and alpha-ketoisocaproic acid or salts thereof in various amounts to a weight per serving of from about 5 g to about 10 g.

17. The composition of claim 1, comprising Threonine and alpha-ketoisocaproic acid or salts thereof in various amounts to a weight per serving of from about 5 g to about 10 g.

18. The composition of claim 1, comprising Glutamine and alpha-ketoisocaproic acid or salts thereof in various amounts to a weight per serving of from about 5 g to about 10 g.

19. The composition of claim 1, comprising Tyrosine and alpha-ketoisocaproic acid or salts thereof in various amounts to a weight per serving of from about 5 g to about 10 g.

20. The composition of claim 1, comprising Cysteine and alpha-ketoisocaproic acid or salts thereof in various amounts to a weight per serving of from about 5 g to about 10 g.

21. The composition of claim 1, comprising Creatine and alpha-ketoglutaminc acid or salts thereof.

22. The composition of claim 1, comprising Creatine and alpha-ketoglutaminc acid or salts thereof in various amounts to a weight per serving of from about 5 g to about 10 g.

23. The composition of claim 1, comprising Creatine and ketoisocaproic acid or salts thereof.

24. The composition of claim 1, comprising Creatine and ketoisocaproic acid or salts thereof in various amounts to a weight per serving of from about 5 g to about 10 g.

25. The composition of claim 1, comprising Glutamic Acid and alpha-ketoisocaproic acid or salts thereof in various amounts to a weight per serving of from about 5 g to about 10 g.

26. The composition of claim 1, comprising Aspartic Acid and alpha-ketoisocaproic acid or salts thereof in various amounts to a weight per serving of from about 5 g to about 10 g.

27. The composition of claim 1, comprising Taurine and alpha-ketoisocaproic acid or salts thereof in various amounts to a weight per serving of from about 5 g to about log.

28. The composition of claim 1, comprising Leucine and ketoisovaleric acid or salts thereof in various amounts to a weight per serving of from about 5 g to about 10 g.

29. The composition of claim 1, comprising Leucine and ketobetamethylvalerate or salts thereof in various amounts to a weight per serving of from about 5 g to about 10 g.

30. The composition of claim 1, comprising Leucine and pyruvate or salts thereof in various amounts to a weight per serving of from about 5 g to about 10 g.

31. The composition of claim 1, comprising Leucine and alpha-ketoglutarate or salts thereof in various amounts to a weight per serving of from about 5 g to about 10 g.

32. The composition of claim 1, comprising Glutamine and alpha-ketoglutarate or salts thereof in various amounts to a weight per serving of from about 5 g to about 10 g.

33. The composition of claim 1, comprising fine-milled Leucine and alpha-ketoisocaproic acid or salts thereof in various amounts to a weight per serving of from about 5 g to about 10 g.

34. The composition of claim 1, comprising Creatine, Alpha Lipoic Acid or salts or esters thereof and alpha-ketoisocaproic acid or salts thereof in various amounts to a weight per serving of from about 5 g to about 10 g.

35. The composition of claim 1 wherein said amino acid and ketoacid are conjugated.

36. The composition of claim 1 wherein the composition is further coated with a layer comprising polyvinyl alcohol, polyethylene glycol, talc, titanium dioxide, riboflavin, colorings, hydroxypropyl cellulose, soy lecithin and polysorbate 80.

37. A method using the composition of claim 5 wherein from about 7.2 g to about 14.4 g of the composition is consumed daily.

38. A method using the composition of claim 6 wherein about 5.4 g of the composition is consumed daily.

39. A method comprising the use of a combination of a monobasic amino acid and a ketoacid to regulate molecular signals to control anabolic activity in skeletal muscles.

40. A method comprising the use of a combination of a monobasic amino acid and a ketoacid to regulate molecular signals to control anti-catabolic activity in skeletal muscles.

41. A method using the composition of claim 1 for stimulating muscle growth.

42. A method using the composition of claim 1 for increasing muscle mass.

43. A method using the composition of claim 1 for increasing muscular performance.

44. A method using the composition of claim 1 for decreasing muscle recovery time.

45. A method using the composition of claim 1 influencing the genetic control system for somatic protein synthesis.

46. The method of claim 37 wherein the amino acid and the ketoacid are conjugated.

47. A method of forming a kit comprising various combinations of a cationic dibasic amino acid, a cationic monobasic amino acid and a ketoacid comprising at least two independent compositions of said kit.

48. The method of claim 47 comprising administering said kit for stimulating muscle growth.

49. The method of claim 47 comprising administering said kit for increasing muscle mass.

50. The method of claim 47 comprising administering said kit for increasing muscular performance.

51. The method of claim 47 comprising administering said kit for decreasing recovery time.

52. The method of claim 47 comprising administering said kit to regulate molecular signals to control anabolic activity in skeletal muscles.

53. The method of claim 47 comprising administering said kit to regulate molecular signals to control anti-catabolic activity in skeletal muscles.

54. The method of claim 47 comprising administering said kit to regulate the genetic control system for somatic protein synthesis.
